(19) United States
(12) Patent Application Publication
(10) Pub. No.: US 2009/0221666 A1
(43) Pub. Date: Sep. 3, 2009

(54) STILBENE DERIVATIVES AND METHODS OF INHIBITING CANCER CELL GROWTH AND MICROBIAL GROWTH

(76) Inventors: George R. Pettit, Paradise Valley, AZ (US); Collin R. Anderson, Latham, NY (US)

Correspondence Address: MCANDREWS HELD & MALLOY, LTD, 500 WEST MADISON STREET, SUITE 3400, CHICAGO, IL, 60661, US

(21) Appl. No.: 11/913,817

(22) PCT Filed: May 12, 2006
(86) PCT No.: PCT/US06/18231

§ 371 Date: Jun. 3, 2008

Related U.S. Application Data
(60) Provisional application No. 60/680,289, filed on May 12, 2005.

(51) Int. Cl. A61K 31/4025; C07D 405/12; A61P 35/00; A61P 35/02; A61P 31/04

(52) U.S. Cl. 514/422; 548/525

(57) ABSTRACT

The present invention provides stilbene derived compounds having antineoplastic and/or antimicrobial activity. Preferred compounds of the invention include compounds of the formula (I) wherein R is Dap, Dap-Dil, Dap-Dil-Val, or Dap-Dil-Val-Dov; R₁ is H, OH, or PO₃Na₂; and R₂ and R₃ are jointly —CH₂— or each independently H, OH, CH₃, or PO₃Na₂; and compounds of the formulas (II), (III) and salts thereof, wherein R and R₁ are independently H or P(O)(OH). The present invention is further directed to methods of inhibiting cancer cell growth and/or microbial growth and compositions for use therewith.

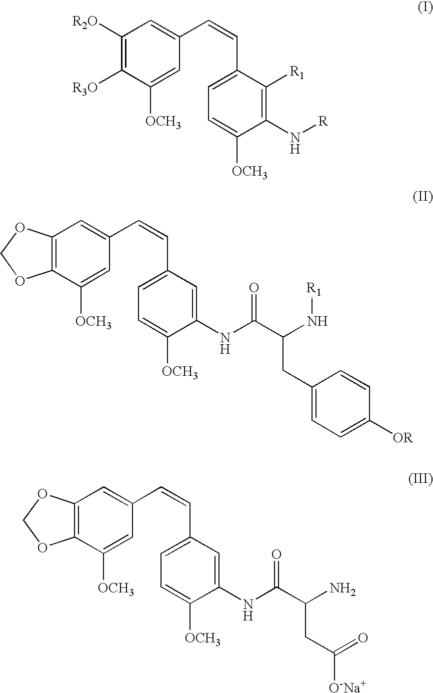

### CROSS-REFERENCE TO RELATED APPLICATIONS

This application is based on and claims the priority to U.S. Provisional Patent Application No. 60/680,289 filed on May 12, 2005, the disclosure of which is incorporated herein in its entirety.

### STATEMENT OF FEDERALLY SPONSORED RESEARCH

Financial assistance for this invention was provided by the United States Government, through the National Institute of Health, Grant Number 5R01 CA090441. Therefore, the United States Government may own certain rights to this invention.

### FIELD OF THE INVENTION

The present invention is directed to stilbene derived compounds having antineoplastic and/or antimicrobial activity. The present invention is further directed to methods of inhibiting cancer cell growth and/or microbial growth in a host inflicted therewith by administering the stilbene derived compounds to the inflicted host.

### BACKGROUND OF THE INVENTION

We recently completed syntheses of 3′-amino derivatives (1a-b, 1d-j) of combretastatin A-2 (Ref. 1) (2c), encouraged by the remarkable success of the potent cancer vascular targeting (Ref. 2-4) that occurs with combretastatin A-4 phosphate prodrug (CA4P) 2h (Ref. 5-10) (see structures below). Subsequently, we undertook herein the synthesis and initial anticancer evaluation of two phosphate derivatives (3a,b) of tyrosine stilbene amide 1i. Attachment of the phosphate group at the 3′-phenol position has been investigated in detail in the combretastatin A and B series (Ref. 1, 11-13). In the present study we chose to use the 4″-phenol group of tyrosine amide 1i for attachment of a phosphate group (3a) as well as obtaining a diphosphoryl derivative (3b) by phosphorylating tyrosine amide 1i at both the amine (Ref. 14) and phenol groups.

A parallel important objective of the present invention involved employing a tetrapeptide segment of dolastatin 10 (4a) for bonding to the 3′-amino group (1b). Dolastatin 10 (D-10, 4a), isolated (Ref. 15-17) from the Indian Ocean sea hare Dolabella auricularia, has continued to progress through preclinical (Ref. 18) and clinical development as an impressive antineoplastic agent (Ref. 19-22). Dolastatin 10 (4a) and various synthetic derivatives also have specific antifungal activity against Cryptococcus neofommans (Ref. 23-26). Previous SAR studies have confirmed that D-10 (4a) inhibits microtubule assembly by binding to the β-tubulin subunit near the vinca site. Additional SAR studies have shown the des-Doe tetrapeptide unit (4b, Dov-Val-Dil-Dap) to be necessary for potent anticancer activity (Ref. 25-26). We report herein the successful synthesis of stilbene derivatives comprising Dov-Val-Dil-Dap (4b).

### SUMMARY OF THE INVENTION

The present invention is directed to certain new stilbene derivatives having antineoplastic activity against cancerous cell lines and/or antimicrobial activity. The present invention provides compounds having the formula

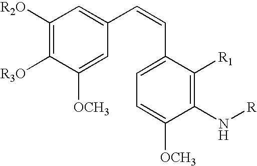

or a salt thereof, wherein R is Dap, Dap-Dil, Dap-Dil-Val, or Dap-Dil-Val-Dov; R₁ is H, OH, or PO₃Na₂; and R₂ and R₃ are jointly —CH₂— or each independently H, OH, CH₃, or PO₃Na₂. In a preferred embodiment, R is Dap or Dap-Dil-Val-Dov and R₁ is H.

In another embodiment, the invention relates to compounds of the formula

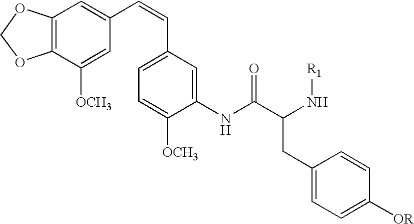

and salts thereof, wherein R and R₁ are independently H or P(O)(OH). In this embodiment, R is preferably P(O)(OH).

In yet another embodiment, the invention provides the compound

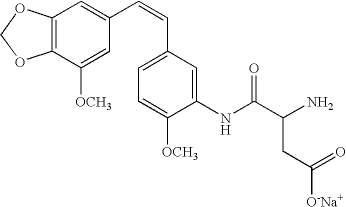

The present invention is further directed to methods of inhibiting cancer cell growth and/or microbial growth. In a preferred embodiment, the method comprises administering to a host inflicted with cancer or a microbial infection at least one stilbene derivative disclosed herein. The compound is typically in a therapeutically effective amount sufficient to inhibit the cancer cell growth or microbial growth in the host.

The present invention also encompasses pharmaceutical compositions. The compositions of the present invention comprise at least one stilbene derivative compound disclosed herein and a pharmaceutically acceptable carrier.

Accordingly, a prime objective of the present invention is to provide stilbene derived compounds that have improved neoplastic activity. A preferred objective is to provide a stilbene derived compound having the ability to bind both the vinca domain and colchicine site of tubulin. A further objective of the present invention is to provide stilbene derived compounds having antimicrobial activity, preferably a broad spectrum of antimicrobial activity.

### DETAILED DESCRIPTION OF THE INVENTION

As described above, the present invention is directed to new stilbene derivatives. Preferably the new stilbene derivatives have antineoplastic activity and/or antimicrobial activity. Specifically, in one embodiment of the present invention the stilbene derivatives have the formula,

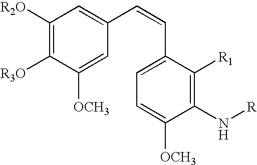

wherein R is Dap, Dap-Dil, Dap-Dil-Val, or Dap-Dil-Val-Dov; R₁ is H, OH, or PO₃Na₂; and R₂ and R₃ are jointly —CH₂— or each independently H, OH, CH₃, or PO₃Na₂. In preferred embodiments, R is Dap and R₁ is H; R is Dap-Dil-Val-Dov and R₁ is H; and R is Dap or Dap-Dil-Val-Dov and R₂ and R₃ are CH₃.

Reference to a compound of the invention herein is understood to include reference to salts thereof, unless otherwise indicated. The term “salt(s)”, as employed herein, denotes acidic and/or basic salts formed with inorganic and/or organic acids and bases. Salts of the compounds of the invention may be formed, for example, by reacting the compound with an amount of acid or base, such as an equivalent amount, in a medium such as one in which the salt precipitates or in an aqueous medium followed by lyophilization. Formation salts is well within the ability of one skilled in the art. Examples of specific salts of the compound of the invention are provided herein, but are not intended to be limiting.

Preferred compounds of the invention inhibit the growth of cancer cells and/or parasitic microbial growth. In a preferred embodiment, the compounds have dual targeting activity, for example, the compounds target both the colchicine and vinca regions of tubulin. Preferably the compound inhibits cancer cells selected from the group consisting of leukemia, pancreas, breast, CNS, lung-NSC, colon, or prostate cancer.

In another embodiment, a method is provided for inhibiting the growth of cancer cells in a host. The method comprises administering to a host inflicted with cancer at least one stilbene derivative disclosed herein. Preferably the compound is administered in a pharmaceutical composition. Preferred pharmaceutical compositions are discussed in detail below. The compound administered is typically in a therapeutically effective amount sufficient to inhibit the cancer cell growth in the host. The host is preferably an animal, more preferably a mammal, and most preferably a human.

In certain embodiments, the method comprises administering to a host in need thereof an effective amount of a compound of the invention and at least one additional therapeutic agent. In one embodiment, the additional therapeutic agent is a chemotherapeutic agent including, but not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, and mixtures thereof.

In another embodiment, the invention is directed to a method of inhibiting microbial growth, preferably in a host as described above. The method comprises administering to a host at least one stilbene derivative disclosed herein in a therapeutically effective amount sufficient to inhibit the microbial growth in the host. The compounds of invention can be administered, alone or in combination with one or more additional antimicrobial agents, to treat microbial infections such as fungal infections and bacterial infections, or combinations of such infections.

Accordingly, in one specific embodiment the invention is a method for treating a microbial infection wherein the fungal infection is resistant to, or sensitive to, an azole antifungal agent, such as fluconazole. The methods of the invention may further include co-administration of a second antimicrobial agent, resulting in administration of an additional antifungal agent and/or an antibacterial agent.

Fungal infections include fungal infections (mycoses), which may be cutaneous, subcutaneous, or systemic. Superficial mycoses include tinea capitis, tinea corporis, tinea pedis, onychomycosis, perionychomycosis, pityriasis versicolor, oral thrush, and other candidoses such as vaginal, respiratory tract, biliary, eosophageal, and urinary tract candidoses. Systemic mycoses include systemic and mucocutaneous candidosis, cryptococcosis, aspergillosis, mucormycosis (phycomycosis), paracoccidioidomycosis, North American blastomycosis, histoplasmosis, coccidioidomycosis, and sporotrichosis. Fungal infections include opportunistic fungal infections, particularly in immunocompromised patients such as those with AIDS. Fungal infections contribute to meningitis and pulmonary or respiratory tract diseases.

Pathogenic organisms include dermatophytes (e.g., Microsporum canis and other M. spp.; and Trichophyton spp. such as T. rubrum, and T. mentagrophytes), yeasts (e.g., Candida albicans, C. Tropicalis, or other Candida species), Torulopsis glabrata, Epidermophyton floccosum, Malassezia fuurfur (Pityropsporon orbiculare, or P. ovale), Cryptococcus neoformans, Aspergillus fumigatus, and other Aspergillus spp., Zygomycetes (e.g., Rhizopus, Mucor), Paracoccidioides brasiliensis, Blastomyces dermatitides, Histoplasma capsulatum, Coccidioides immitis, and Sporothrix schenckii. Fungal infections include Cladosporium cucumerinum, Epidermophyton floccosum, and Microspermum ypseum. Examples of current antimycotic drugs include nystatin, clotrimazole, amphotericin B, ketoconazole, fluconazole, and itraconazole.

Bacterial infections result in diseases such as bacteremia, pneumonia, meningitis, osteomyelitis, endocarditis, sinusitis, arthritis, urinary tract infections, tetanus, gangrene, colitis, acute gastroenteritis, bronchitis, and a variety of abscesses, nosocomial infections, and opportunistic infections. Bacterial pathogens include Gram-positive cocci such as Staphylococcus aureus, Streptococcus pyogenes (group A), Streptococcus spp. (viridans group), Streptococcus agalactiae (group B), S. bovis, Streptococcus (anaerobic species), Streptococcus pneumoniae, and Enterococcus spp.; Gram-negative cocci such as Neisseria gonorrhoeae, Neisseria meningitidis, and Branhamella catarrhalis; Gram-positive bacilli such as Bacillus anthracis, Corynebacterium diphtheriae and Corynebacterium species which are diptheroids (aerobic and anerobic), Listeria monocytogenes, Clostridium tetani, Clostridium difficile, Escherichia coli, Enterobacter species, Proteus mirablis and other spp., Pseudomonas aeruginosa, Klebsiella pneumoniae, Salmonella, Shigella, Serratia, and Campylobacter jejuni.

Preferably the compound used inhibits a microbe from the genus Neisseria, Enterococcus, Streptococcus or Cryptococcus and even more preferably the compound inhibits a microbe selected from the group consisting of: Neisseria gonorrhoeae; Enterococcus faecalis; Streptococcus pneumoniae; and Cryptococcus neoformans.

Pharmaceutical Compositions and Dosage Forms

Pharmaceutical compositions can be used in the preparation of individual dosage forms. Consequently, pharmaceutical compositions and dosage forms of the invention comprise the active ingredients disclosed herein. The notation of “active ingredient” signifies the compounds of the invention described herein or salts thereof. Pharmaceutical compositions and dosage forms of the invention can further comprise a pharmaceutically acceptable carrier.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which an active ingredient is administered. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. In addition, other excipients can be used.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a neoplastic disease or microbial infection may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

As used herein, a “therapeutically effective amount” is an amount sufficient to either inhibit (partially or totally) formation of a tumor or a hematological malignancy or to reduce its further progression or to inhibit the growth of a microbe of interest. For a particular condition or method of treatment, the dosage is determined empirically, using known methods, and will depend upon facts such as the biological activity of the particular compound employed, the means of administrations, the age, health and body weight of the host; the nature and extent of the symptoms; the frequency of treatment; the administration of other therapies and the effect desired. Hereinafter are described various possible dosages and methods of administration with the understanding that the following are intended to be illustrative only. The actual dosages and method of administration or delivery may be determined by one of skill in the art.

Typical illustrative dosage forms of the invention comprise a compound or mixture of compounds of the invention thereof as an active ingredient in an amount of from about 1 mg to about 2000 mg, more preferably from about 25 mg to about 1000 mg, even more preferably from about 50 mg to about 750 mg, and most preferably from about 100 mg to about 500 mg.

For illustrative purposes, dosage levels of the administered active ingredients may be: intravenous, 0.01 to about 20 mg/kg; intramuscular, 0.1 to about 50 mg/kg; orally, 0.05 to about 100 mg/kg; intranasal instillation, 0.5 to about 100 mg/kg; and aerosol, 0.5 to about 100 mg/kg of host body weight.

Expressed in terms of concentration, an active ingredient may be present in the compositions of the present invention for localized use about the cutis, intranasally, pharyngolaryngeally, bronchially, intravaginally, rectally, or ocularly in concentration of from about 0.01 to about 50% w/w of the composition; preferably about 1 to about 20% w/w of the composition; and for parenteral use in a concentration of from about 0.05 to about 50% w/v of the composition and preferably from about 5 to about 20% w/v.

The active ingredients to be employed as antineoplastic or antimicrobial agents can be easily prepared in such unit dosage form with the employment of pharmaceutical materials which themselves are available in the art and can be prepared by established procedures. The following preparations are illustrative of the preparation of dosage forms of the present invention, and not as a limitation thereof.

Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A preferred solid oral dosage form of the invention comprises an active ingredient, anhydrous lactose, microcrystalline cellulose, polyvinylpyrrolidone, stearic acid, colloidal anhydrous silica, and gelatin.

Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

Parenteral Dosage Forms

Parenteral dosage forms can be administered to patients by various routes including, but not limited to, subcutaneous, intravenous, bolus injection, intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts of the active ingredients can be used to further adjust the properties of the resulting composition.

The present invention will now be illustrated by the following non-limiting examples.

### EXAMPLES

### Synthesis of Stilbene Derivatives

Our synthesis of the tyrosine stilbene amide 1i (Ref. 27) presented the opportunity to develop two additional phosphate prodrugs 3a and 3b in the combretastatin series (See chemical structures below).

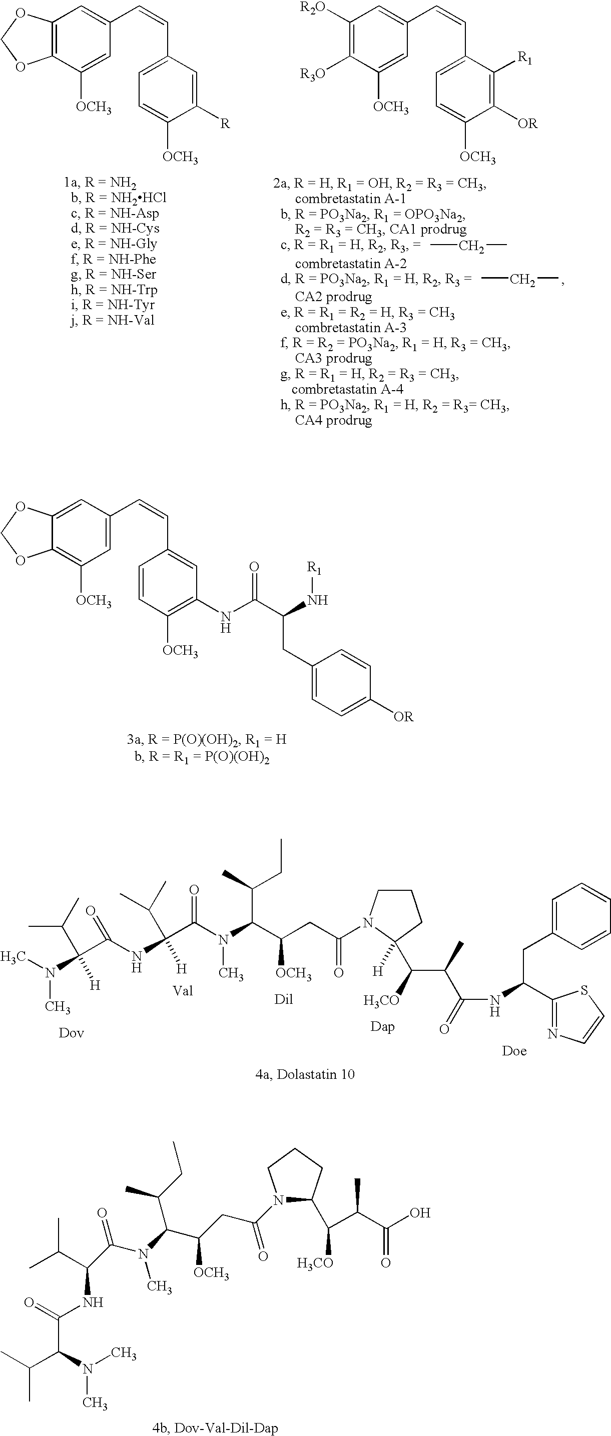

Accordingly, the 4″-phenol and α-amino groups were phosphorylated in one step using dibenzyl phosphite (1i→5) (Scheme 1).

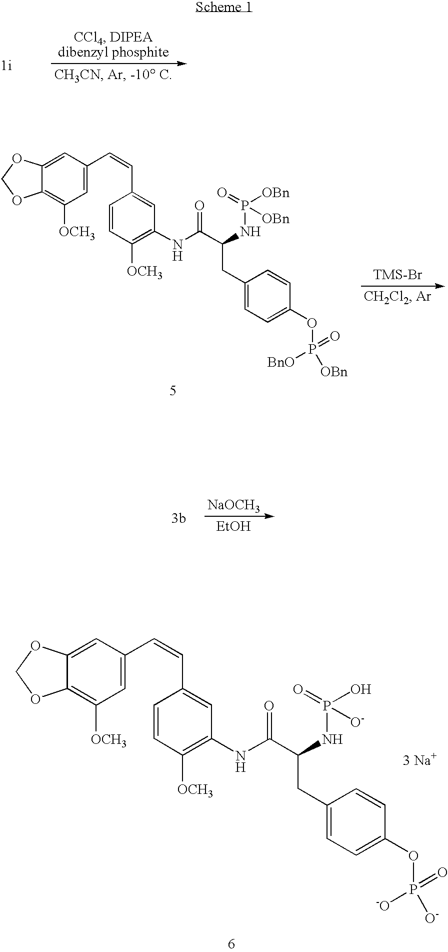

Debenzylation of the resulting phosphate diesters (5) was achieved using bromotrimethylsilane to afford the free acid (3b) which was subsequently converted to sodium salt 6 using sodium methoxide in methanol (Ref. 11-12). Phosphorylation solely at the 4″-phenol position was accomplished via the coupling of the 3′-amino stilbene 1a to O-tert-butyl-N^{α}-Z-L-Tyr using PyBroP as the coupling reagent (Ref. 28) to provide amide 7 (Scheme 2).

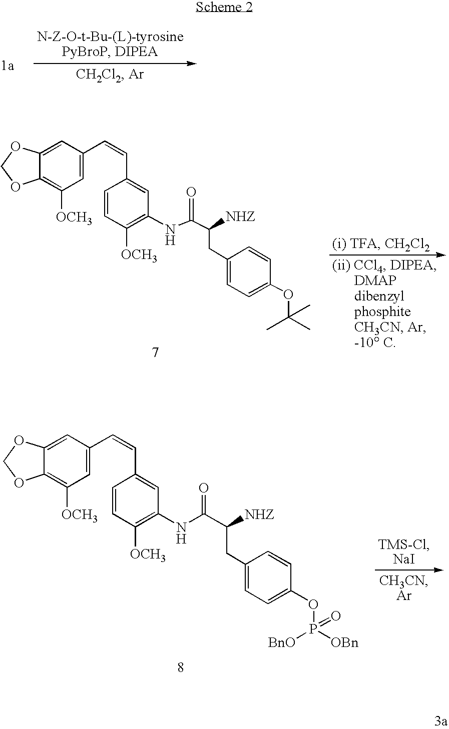

tert-Butyl removal using TFA in DCM followed by phosphorylation afforded the dibenzyl ester 8. Debenzylation at the ester and benzyloxycarbonyl removal at the amine were achieved simultaneously with the in situ generation of trimethylsilyl iodide (TMSI) from the reaction of sodium iodide and chlorotrimethylsilane in acetonitrile (Ref. 29-30). This method resulted in the immediate precipitation of the phosphate prodrug 3a. Surprisingly, both phosphoric acids and the corresponding sodium salts proved to be sparingly soluble in water. We further investigated the possibility of making a soluble amide derivative by first making the hydrochloride salt of 1i, (Scheme 3) and second by coupling amine 1a to aspartic acid and converting to the β-aspartate sodium salt 11 (Scheme 4).

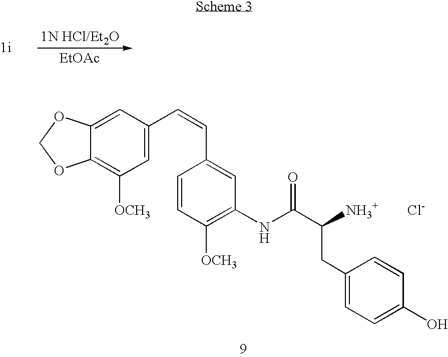

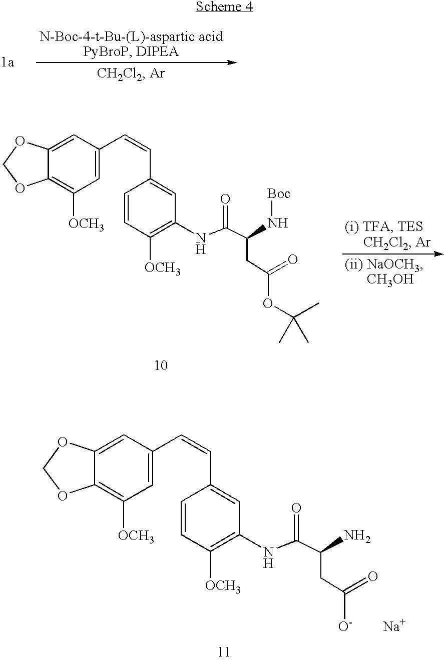

Amide formation was accomplished using PyBroP followed by removal of the β-tert-butyl protecting group in the presence of triethylsilane (Ref. 31). The resulting carboxylic acid was subsequently treated with sodium methoxide in methanol yielding sodium salt 11. Both the hydrochloride salt 9 and the sodium salt 11 were essentially insoluble (<0.1 mg/mL) in water. Although we found earlier that the combretastatin A-2 phosphate prodrug (2d) has reduced aqueous solubility when compared to its combretastatin A-1 (2b) and A-3 counterparts (2f) (Ref. 11-12), we were surprised to find that our new CA2 modifications (1b, 3a, 6, 9, and 11) exhibited only sparing water solubility presumably owing to the hydrophobicity created by the stilbene (Table 1). Interestingly, addition of the phosphate group to the tyrosine amide 1i to yield 3a resulted in decreased anticancer activity (Table 2).

TABLE 1
Solubility (mg/mL) of various combretastatin prodrugs.

| | Compound | | | | | | | | |
|---|---|---|---|---|---|---|---|---|---|
| | 1a | 2b | 2d | 2f | 2h | 3a | 6 | 9 | 11 |
| Solubility^{a} | <0.1 | 120 | 18 | >218 | 20 | <0.1 | <0.1 | <0.1 | <0.1 |

^{a}Solubility values were obtained using 1 mL distilled water at 25° C.

TABLE 2
Human cancer cell line GI₅₀ (μg/mL) and murine P388 lymphocytic leukemia cell
line inhibitory activity ED₅₀ (μg/mL) of the 3¹-substituted stilbenes.

| | | | | | Lung- | | | |
|---|---|---|---|---|---|---|---|---|
| | | | | CNS | NSC | | | |
| | Leukemia | Pancreas | Breast | SF- | NCI- | Colon | Prostate | |
| Compound | P388 | BXPC-3 | MCF-7 | 295 | H460 | KM20L2 | DU145 | Mean |
| 1a | 0.00189 | 0.0071 | 0.0047 | 0.023 | 0.0050 | 0.022 | 0.028 | 0.013 |
| 3a | >10 | >1 | >1 | >1 | >1 | >1 | >1 | — |
| 5 | >10 | — | — | — | — | — | — | — |
| 6 | 0.270 | 18.6 | 1.3 | >1 | >1 | >1 | >1 | — |
| 7 | 0.380 | 0.049 | 0.36 | 0.44 | 0.41 | 1.8 | 0.39 | 0.61 |
| 8 | 0.133 | >10 | >10 | >10 | >10 | >10 | >10 | — |
| 9 | 0.0329 | 0.064 | 0.041 | 0.064 | 0.096 | 0.034 | 0.080 | 0.059 |
| 10 | 0.385 | 3.4 | 2.4 | 1.7 | 3.8 | 2.3 | 3.0 | 2.4 |
| 11 | 0.361 | 0.38 | 0.51 | 0.25 | 0.30 | 0.19 | 0.33 | 0.33 |
| 13 | 0.225 | 0.58 | 0.53 | 0.75 | 0.52 | 1.9 | 0.38 | 0.70 |
| 14 | 0.0263 | 0.23 | 0.054 | 0.062 | 0.083 | 0.26 | 0.070 | 0.11 |
| 16 | <0.01 | 0.048 | 0.0031 | 0.025 | 0.033 | 0.020 | 0.032 | — |

Since the cancer cell line results obtained for each of the substances recorded in Table 2 were obtained in solution, presumably the sparing water solubility did not significantly influence the cell line results and effectiveness. The reduced ability to hydrogen bond in vitro could be important however.

The des-Doe tetrapeptide unit (4b) of the potent anticancer drug dolastatin 10 (D-10, 4a) isolated in 1984 (Ref. 15) was chosen for coupling with amine 1a. By replacing the Doe portion of D-10 with the amine 1a, we planned to synthesize a stilbene peptide with the possibility of binding to the colchicine and/or vinca regions of tubulin. The tripeptide Dov-Val-Dil TFA salt (15) and Boc-Dap (12) were obtained via published methods (Ref. 32,33). PyBroP coupling of amine 1a to Boc-Dap (12) followed by Boc deprotection with TFA in DCM afforded compound 14 (Scheme 5).

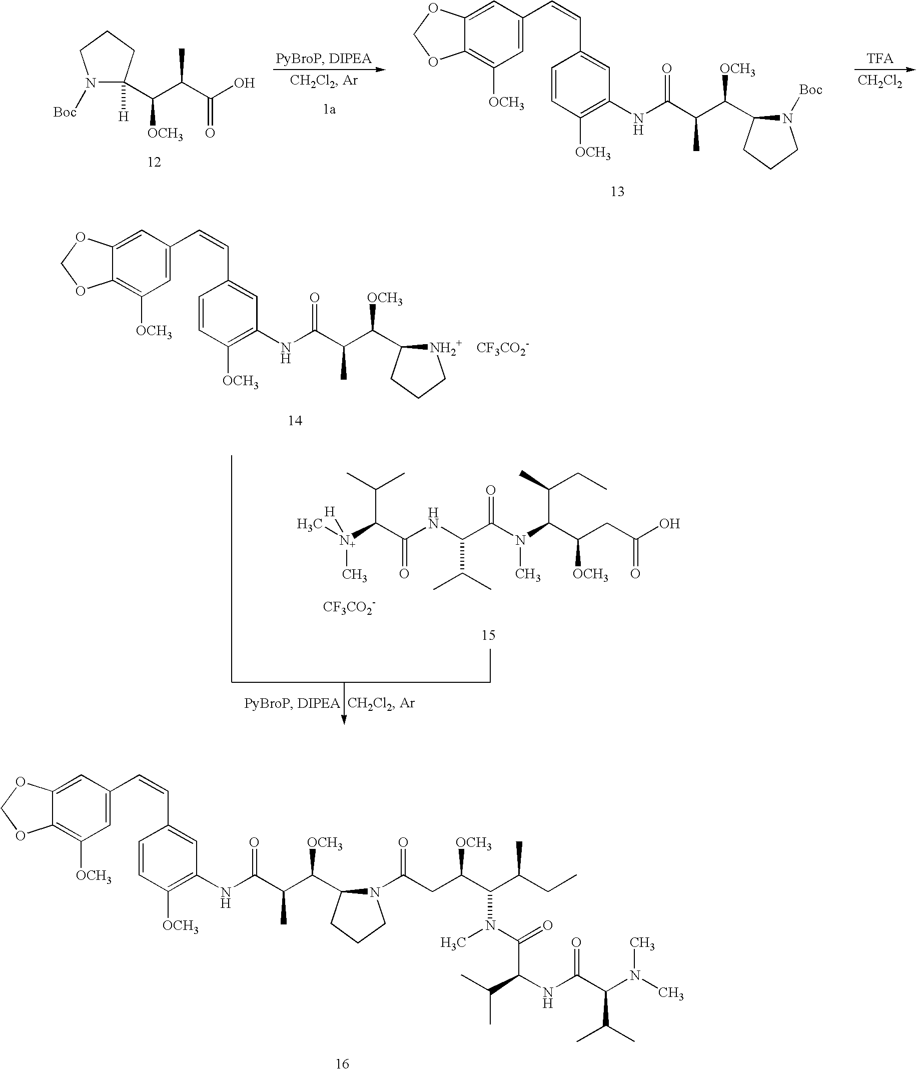

Further coupling resulted in des-Doe D-10 stilbene amide 16. The cancer cell inhibition activity of the potentially dual functioning anticancer agent 16 was notable (Table 2 above), as was the broad spectrum antimicrobial action of amide 14 discussed below.

### Antimicrobial Evaluation of Stilbene Derivatives

Compounds were screened in broth microdilution assays according to National Committee for Clinical Laboratory Standards (Ref. 43-44). Assays were repeated at least twice on separate days. Compound 14 was found to inhibit the growth of Cryptococcus neoformans ATCC 90112 (MIC=64 μg/mL); the pathogenic bacterium Neisseria gonorrhoeae ATCC 49226 (MIC=16 μg/mL); and the opportunistic bacteria Streptococcus pneumoniae ATCC 6303 (MIC=8-16 μg/mL); Enterococcus faecalis ATCC 29212 (MIC=32-64 μg/mL). Prodrug 3a inhibited N. gonorrhoeae ATCC 49226 (MIC=0.125 μg/mL); and E. faecalis ATCC 29212 (MIC=64 μg/mL). Prodrug 6 inhibited N. gonorrhoeae ATCC 49226 (MIC=4 μg/mL). Compound 8 inhibited E. faecalis ATCC 29212 (MIC=64 μg/mL). Compound 13 inhibited C. neoformans ATCC 90112 (MIC=64 μg/mL).

### Antineoplastic Evaluation of Stilbene Derivatives

Previous work showed that D-10 (4a) is an exceptionally cytotoxic antimitotic agent and that it inhibits tubulin assembly by binding tightly in the vinca domain of tubulin (its inhibition of vinblastine binding to tubulin, although extensive, is noncompetitive) (Ref. 34-35). In contrast, while combretastatin A-4 (2g) is quite potent for a colchicine site drug, it is about 100-fold less cytotoxic than D-10 (4a), and the stilbene inhibits tubulin assembly by binding avidly to the colchicine site in a competitive fashion (Ref. 36-37). These findings were confirmed in the studies presented in Table 3 and Table 4 below. Note the similar inhibitory effects of 4a and 2g on tubulin assembly, their contrasting effects on inhibition of binding of radiolabeled vinblastine and colchicine, and the greater than 100-fold enhanced inhibition of growth of the MCF-7 cells by 4a relative to 2g.

TABLE 3
Effects of Peptide 16 and Related Compounds on Tubulin Assembly,
Binding of Colchicine and Vinblastine to Tubulin.

| | Inhibition | Inhibition | | Inhibition | |
|---|---|---|---|---|---|
| | of tubulin | of colchicine | | of vinblastine | |
| | assembly | binding % | | binding % | |
| | IC₅₀ | Inhibition ± SD^{a} | | Inhibition ± SD^{a} | |
| Compound | (μM) ± SD^{a} | 2 μM^{b} | 50 μM^{b} | 10 μM^{b} | 80 μM^{b} |
| 16 | 1.3 ± 0.04 | | 30 ± 2 | 48 ± 3 | 87 ± 2 |
| 4b | 1.4 ± 0.03 | | 5.2 ± 3 | 16 ± 6 | 68 ± 0.4 |
| 1b | 3.4 ± 0.5^{d} | 85 ± 3 | | 0 | 0 |
| 4a | 0.70 ± 0.06 | 13 ± 5 | | 96 ± 0.7 | |
| 4b + 1b^{c} | | | | | |
| 2g | 1.8 ± 0.3 | 98 ± 0.4 | | 0 | 0 |

^{a}SD, Standard deviation.
^{b}Inhibitor concentrations.
^{c}Equimolar concentrations of compounds 4b and 1b were present in all cell culture mixtures.
^{d}Compound 1a had a nearly identical IC₅₀ value (4.3 ± 0.5 μM).

TABLE 4
Effects of Peptide 16 and Related Compounds on the Growth
and Mitotic Index of MCF-7 Breast Cancer Cells

| | Inhibition of growth | Mitotic index |
|---|---|---|
| | of MCF-7 breast cancer | of MCF-7 cells |
| Compound | cells IC₅₀ (nM) ± SD^{a} | % Mitoses^{b} |
| 16 | 17 ± 7 | 57 |
| 4b | 36 ± 10 | 56 |
| 1b | 68 ± 30 | 65 |
| 4a | 0.083 ± 0.06 | 39 |
| 4b + 1b^{c} | 14 ± 2 | 52 |
| 2g | 11 ± 10 | 46 |

^{a}SD, Standard deviation.
^{b}The mitotic indices were determined following growth of the cells for 16 h at 10 times the IC₅₀ concentration. Without drug, 2.9% of the cells were in mitosis.
^{c}Equimolar concentrations of compounds 4b and 1b were present in all cell culture mixtures.

Previous SAR studies with D-10 (Ref. 24) (4a) structural modifications had shown that loss of its C-terminal unit Doe had a significant effect on cell growth, and a greater effect on inhibition of ligand binding to tubulin than on inhibition of tubulin assembly. Thus, Dov-Val-Dil-Dap (4b) is only 2-fold less active than 4a as an assembly inhibitor, but at least 6-fold less active as an inhibitor of MCF-7 cell growth (Table 4). These SAR studies had also shown that replacement of the Doe residue with a variety of aromatic derivatives could restore some or all of the activity of D-10 (4a) in both the cytological and biochemical assays (Ref. 23-24).

SAR studies with structural manipulation of combretastatin A4 (Ref. 27, 36) (2g) revealed that relatively minor effects on cytotoxicity and tubulin interactions were observed when a methylenedioxy bridge replaced two vicinal methoxy groups in the A ring or an amino group replaced the hydroxyl group in the B ring. These findings are demonstrated again in Tables 3 and 4, where amine hydrochloride 1b, incorporating both these changes, is compared with CA4 2g. In addition, we found that a variety of amino acid amides formed from amine 1a had sharply reduced activity in the tubulin-based biochemical assays but retained the amine's cytotoxic activity (Ref. 27). Since these compounds caused a marked increase in the mitotic index of drug-treated cells, the most reasonable explanation was that the amine 1a was regenerated by either extracellular or intracellular hydrolysis of the amides.

When the dolastatin 10-combretastatin amine hybrid 16 was evaluated, it was found to be active in all the biochemical assays (Tables 3 and 4), although its effect on the binding of [³H]colchicine was relatively weak, concordant with the weak activities observed previously with the amino acid amides of 1a (Ref. 27). In contrast, compound peptide 16 was about half as active as D-10 (4a) as an inhibitor of both tubulin assembly and [³H]vinblastine binding to tubulin. It appears that peptide 16 is likely acting primarily as a D-10 (4a) analog, however, dual activity having combretastatin analog has unique benefits and properties not provide by prior art stilbene compounds.

Using MCF-7 cells for a detailed comparison, we found that peptide 16 was about twice as active as Dov-Val-Dil-Dap (4b) and 4 times as active as hydrochloride 1b, but only 1/200^{th} as active as D-10 (4a). Further, when 4b and 1b were mixed in equimolar amounts, as would occur if 16 were completely hydrolyzed, an IC₅₀ value for inhibition of the growth of the MCF-7 cells was obtained that was almost identical to that obtained with peptide 16 (14 vs. 17 nM). Since the IC₅₀ values obtained for 16, 4b, and 1b are in the same range, it is impossible to determine whether the cytotoxicity of 16 derives from its acting primarily as a relatively weak D-10 (4a) analog or primarily as a relatively potent combretastatin A-4 (2g) analog. The similarity in IC₅₀ values obtained with 16 and with the mixture of its components 4b and 1b indicates that it, like the previously studied amides (Ref. 27), undergoes at least partial extracellular or intracellular hydrolysis. Finally, the similarity in IC₅₀ values for 16, 4b, 1b, and the 4b+1b mixture indicates that there is little synergistic obvious in vitro cytotoxic effect obtained from having 4b and 1b conjoined in a single molecule. Compound 16 and similar stilbene derivatives may however have benefits with respect to effectiveness, administration, dosage, and in vivo delivery.

In conclusion, although initial attempts to synthesize a very water soluble derivative from the 3,4-methylenedioxy-5,4′-dimethoxy-3′-amido-Z-stilbene tyrosine amide and aspartate amide series proved unrewarding, three new stilbene derivatives (9, 14, and 16) strongly inhibiting a mini-panel of human cancer cell lines were discovered as discussed above. Most importantly, the preliminary biological results from the potentially multi-targeting compounds (amide 14 and peptide 16) merit further investigation as to the broad spectrum antimicrobial activity and dual anticancer activity.

### Materials and Methods:

Ether refers to diethyl ether and Ar to argon gas. Bromo-tris-pyrrolidino-phosphonium hexafluorophosphate (PyBroP), O^{β}-tert-butyl-N^{α}-Boc-L-aspartic acid, and O-tert-butyl-N^{α}-Z-L-tyrosine were obtained from Calbiochem-Novabiochem Corporation (San Diego, Calif.). Diisopropylethylamine (DIPEA), anhydrous methanol, sodium methoxide, triethylsilane (TES), and trifluoroacetic acid (TFA) were obtained from Acros Organics (Fisher Scientific, Pittsburgh, Pa.). All other reagents were purchased from Sigma-Aldrich Chemical Company (Milwaukee, Wis.).

Reactions were monitored by thin layer chromatography using Analtech silica gel GHLF Uniplates visualized under short-wave UV irradiation. Solvent extracts of aqueous solutions were dried over anhydrous magnesium sulfate or sodium sulfate. Where appropriate, the crude products were separated by column chromatography on flash (230-400 mesh ASTM) silica from E. Merck, gravity (70-230 mesh ASTM) silica from E. Merck, or Sephadex LH-20.

Melting points are uncorrected and were determined employing an Electrothermal 9100 apparatus. Optical rotations were measured using a Perkin-Elmer 241 polarimeter. The [α]_{D}-values are given in 10⁻¹ deg cm² g⁻¹. The ¹H- and ¹³C-NMR spectra were recorded employing Varian Gemini 300, Varian Unity 400, or Varian Unity 500 instruments using a deuterated solvent and were referenced either to TMS or the solvent. HRMS data were recorded with a JEOL LCmate mass spectrometer. Elemental analyses were determined by Galbraith Laboratories, Inc., Knoxville, Tenn.

### 3,4-Methylenedioxy-5,4′-dimethoxy-3′-[O,N^{α}-di(bis-benzylphosphoryl)-L-Tyr]-amido-Z-stilbene (5)

To a stirred solution of amine 1i (Ref. 27) (71 mg, 0.15 mmol) in acetonitrile (1 mL) at −10 EC in an acetone/ice bath, under Ar, was added carbon tetrachloride (0.15 ml, 1.6 mmol, 11 eq). Ten minutes later DIPEA (0.12 mL, 0.66 mmol, 4.4 eq) and 4-dimethylaminopyridine (3.8 mg, 0.031 mmol, 0.21 eq) were added, followed 1 min later by the dropwise addition of dibenzylphosphite (0.11 mL, 0.47 mmol, 3.1 eq). After 1 h, the reaction was warmed to rt and aqueous 0.5 M KH₂PO₄ was added. The mixture was extracted with EtOAc (3×10 mL), and the combined extracts were washed with brine (15 mL) and water (15 mL). Upon drying and condensing in vacuo, the product was separated by gravity column chromatography (4:1, DCM-EtOAc) to afford a colorless oil 5 (97 mg, 65%): R_{f} 0.12 (4:1, DCM:EtOAc); [α]²⁵_{D} −45.1E (c 0.72, CHCl₃); P-NMR (400 MHz, CDCl₃) δ 5.693, −8.113; ¹H-NMR (400 MHz, CDCl₃) δ 2.89 (1H, m, —CH₂—), 3.18 (1H, m, —CH₂—), 3.56 (3H, s, OCH₃), 3.71 (3H, s, OCH₃), 4.05 (1H, m, alpha H), 4.96 (8H, m, 4×—CH₂—), 5.87 (2H, s, —OCH₂O—), 6.39 (1H, d, J=12.0 Hz, vinyl H), 6.45 (3H, m, vinyl H, 2×ArH), 6.59 (1H, d, J=8.4 Hz, ArH), 6.97 (1H, dd, J=8.4, 1.6 Hz, ArH), 6.99 (2H, d, J=8.4 Hz, 2×ArH), 7.03 (2H, d, J=8.8 Hz, 2×ArH), 7.29 (20H, m, 20×ArH), 8.27 (1H, d, J=1.6, ArH), 8.36 (1H, s); and HRMS calcd for C₅₄H₅₃N₂O₁₂P₂ [M+H]⁺ 983.3074; found 983.3115. Anal. calcd for C₅₄H₅₂N₂O₁₂P₂; C, 65.98; H, 5.33; N, 2.85. Found: C, 65.51; H, 5.55; N, 2.84.

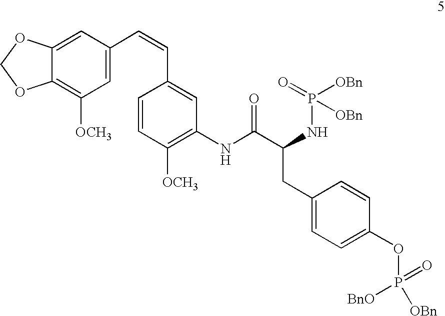

### Sodium 3,4-methylenedioxy-5,4′-dimethoxy-3′-(L-Tyr)-amido-Z-stilbene 3′-O,N^{α}-diphosphate (6)

To a stirred solution of benzyl phosphate 5 (76 mg, 0.077 mmol) in DCM (1 mL) at 0 EC, under Ar, was added bromotrimethylsilane (35 μL, 0.33 mmol, 4.3 eq). The reaction mixture was stirred for 30 min and concentrated under vacuum. The residue was dissolved in EtOH (1 mL), and sodium methoxide (20 mg, 0.36 mmol, 4.7 eq) was added, yielding a precipitate that was collected and washed with EtOAc and ether. The product (6) was obtained as a colorless powder (52 mg, 95%); m.p. (dec.) 188-190 EC; [α]_{D} −30.6E (c 0.72, DMSO); P-NMR (400 MHz, DMSO) δ 6.262, 0.585; ¹³C-NMR (300 MHz, DMSO) δ 143.8, 132.3, 130.4, 130.2, 129.7, 129.4, 127.4, 127.2, 126.3, 122.1, 119.7, 116.5, 115.1, 111.0, 106.8, 101.2, 99.5, 56.6, 56.2, 55.9; ¹H-NMR (400 MHz, DMSO) δ 2.63 (1H, m, —CH₂—), 3.10 (1H, m, —CH₂—), 3.30 (1H, d, J=8.8 Hz), 3.56 (1H, m), 3.86 (3H, s, OCH₃), 3.87 (3H, s, OCH₃), 6.00 (2H, s, —OCH₂O—), 6.55 (1H, d, J=0.8 Hz, ArH), 6.99 (8H, m, 2×vinyl H, 6×ArH), 7.11 (1H, d, J=8.4 Hz, ArH), 7.23 (1H, dd, J=6.8, 1.6 Hz, ArH), 10.12 (1H, br s). Anal. calcd for C₂₆H₂₅N₂Na₃O₁₂P₂3H₂O: C, 42.06; H, 4.21; N, 3.77. Found: C, 41.68; H, 4.65; N, 3.45.

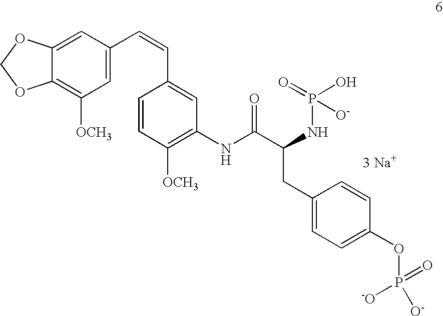

### 3,4-Methylenedioxy-5,4′-dimethoxy-3′-(O-tert-butyl-N^{α}-Z-L-Tyr)-amido-Z-stilbene (7)

To a stirred mixture of amine 1a (0.19 g, 0.63 mmol), O-tert-butyl-N^{α}-Z-L-Tyr (0.45 g, 1.2 mmol, 1.9 eq), and PyBroP (0.57 g, 1.2 mmol, 1.9 eq) in DCM (2 mL) at 0 EC under Ar was added DIPEA (0.55 ml, 3.2 mmol, 5.1 eq) (Ref. 32-33). The reaction mixture was stirred for 45 min at rt and concentrated under vacuum. The product was obtained by gravity column chromatography (4:1, DCM:EtOAc) as a colorless oil (7, 0.38 g, 93%); R_{f} 0.74 (4:1, DCM:EtOAc); [α]²⁴_{D} +1.9E (c 1.04, CHCl₃); ¹H-NMR (300 MHz, CDCl₃) δ 1.30 (9H, s, tBu), 3.05 (2H, m, —CH₂—), 3.63 (3H, s, OCH₃), 3.68 (3H, s, OCH₃), 4.47 (1H, m, alpha H), 5.05 (2H, d, J=2.4 Hz, —CH₂—), 5.60 (1H, br), 5.85 (2H, s, —OCH₂O—), 6.33 (1H, d, J=12.0 Hz, vinyl H), 6.40 (3H, m, vinyl H, 2×ArH), 6.59 (1H, d, J=9.0 Hz, ArH), 6.83 (2H, m, ArH), 6.91 (1H, dd, J=8.1, 1.5 Hz, ArH), 7.05 (2H, d, J=8.4 Hz, 2×ArH), 7.26 (5H, m, 5×ArH), 7.92 (1H, s), 8.22 (1H, d, J=2.1 Hz, ArH); and HRMS calcd for C₃₈H₄₁O₈N₂ [M+H]⁺ 653.2863, found 653.2876. Anal. calcd for C₃₈H₄₀N₂O₈: C, 69.92; H, 6.18; N, 4.29. Found: C, 69.45; H, 6.37; N, 4.21.

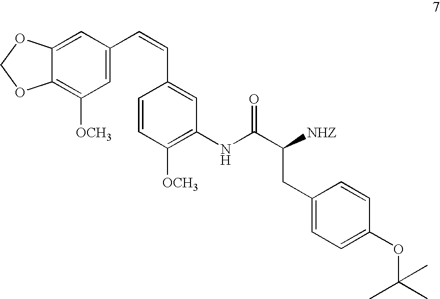

### 3,4-Methylenedioxy-5,4′-dimethoxy-3′-(O-bis-benzylphosphoryl-N^{α}-Z-L-Tyr)-amido-Z-stilbene (8)

To a stirred solution of amide 7 (0.26 g, 0.40 mmol) in DCM (2 mL) was added TFA (2 mL). The reaction mixture was stirred for 20 min, the solution was concentrated under vacuum, and the free phenol obtained by gravity column chromatography (4:1, DCM:EtOAc) was placed in acetonitrile (4 mL) under Ar. The mixture was cooled to −10 EC, and carbon tetrachloride (0.21 ml, 2.1 mmol, 5.3 eq) was added. Ten minutes later DIPEA (0.16 mL, 0.89 mmol, 2.2 eq) and 4-dimethylaminopyridine (5 mg, 0.041 mmol, 0.10 eq) were added, followed 1 min later by the dropwise addition of dibenzyl phosphite (0.15 mL, 0.65 mmol, 1.6 eq). After 2 h the reaction mixture was warmed to rt, and aqueous 0.5 M KH₂PO₄ (16 mL) added. The same procedure given for phosphate 5 was followed to obtain the product 8 as a colorless oil (0.24 g, 71%); R_{f} 0.60 (4:1, DCM:EtOAc); [α]²³_{D} −5.1E (c 0.25, CHCl₃); P-NMR (400 MHz, CDCl₃) δ −8.095; ¹H-NMR (MHz, CDCl₃) δ 3.08 (2H, m, —CH₂—), 3.66 (1H, s, OCH₃), 3.72 (1H, s, OCH₃), 4.51 (1H, m, alpha H), 5.08 (2H, d, J=0.9 Hz, —CH₂—), 5.11 (4H, m, 2×—CH₂—), 5.44 (1H, br d, J=7.2 Hz), 5.90 (2H, s, —OCH₂O—), 6.39 (1H, d, J=12.0 Hz, vinyl H), 6.46 (3H, m, vinyl H, 2×ArH), 6.62 (1H, d, J=8.1 Hz, ArH), 6.98 (1H, dd, J=8.4, 1.8 Hz, ArH), 7.04 (2H, d, J=8.1 Hz, 2×ArH), 7.14 (2H, d, J=8.1 Hz, 2×ArH), 7.31 (15H, m, 15×ArH), 7.90 (1H, s), 8.24 (1H, d, J=2.4 Hz, ArH); and HRMS calcd for C₄₈H₄₆N₂O₁₁P [M+H]⁺857.2840; found 857.2853. Anal. calcd for C₄₈H₄₅N₂O₁₁P; C, 67.28; H, 5.29; N, 3.27. Found: C, 66.89; H, 5.27; N, 3.17.

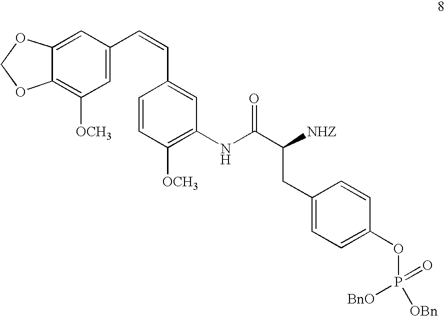

### 3,4-Methylenedioxy-5,4′-dimethoxy-3′-(O-phosphoryl-N^{α}-L-Tyr)-amido-Z-stilbene (3a)

To a stirred solution of benzyl ester 8 (98 mg, 0.11 mmol) in acetonitrile under Ar was added sodium iodide (60 mg, 0.40 mmol, 3.6 eq), followed by chlorotrimethylsilane (51 μL, 0.41 mmol, 3.7 eq). A white precipitate formed while the reaction mixture was stirred for 20 min. Aqueous (1%) sodium thiosulfate (0.5 mL) was added to the mixture before the precipitate was collected and washed with ethyl acetate, water, and acetone. The product was obtained as a colorless amorphous solid (3a, 35 mg, 58%): m.p. (dec.) 175-177 EC; [α]²⁵_{D} −6.3E (c 0.35, DMSO); P-NMR (400 MHz, DMSO) δ −2.043; ¹³C-NMR (400 MHz, DMSO) δ 170.6, 156.1, 149.0, 148.8, 143.3, 136.9, 134.2, 132.3, 129.7, 129.6, 128.9, 128.3, 127.7, 127.2, 127.0, 126.4, 122.8, 119.5, 118.9, 111.3, 106.8, 101.2, 99.5, 65.4, 57.0, 56.2, 36.4; ¹H-NMR (400 MHz, DMSO) δ 2.78 (2H, m, —CH₂—), 3.06 (1H, m, —CH₂—), 3.82 (3H, s, OCH₃), 3.86 (3H, s, OCH₃), 4.49 (1H, m, alpha H), 5.98 (2H, s, —OCH₂O—), 6.89 (1H, d, J=12.8 Hz, vinyl H), 6.94 (1H, s, ArH), 7.07 (2H, m, vinyl H, ArH), 7.22 (1H, d, J=6.4 Hz, ArH), 7.28 (2H, d, J=5.6, 2×ArH), 7.32 (2H, d, J=5.6, 2×ArH), 7.74 (1H, d, J=6.4, ArH), 8.22 (1H, s), 9.25 (1H, s).

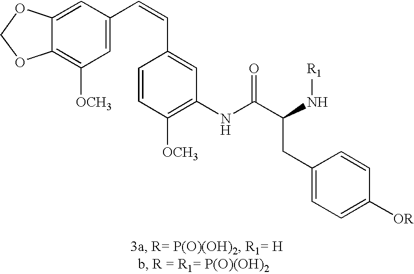

### 3,4-Methylenedioxy-5,4′-dimethoxy-3′-(N^{α}-L-Tyr)-amido-Z-stilbene hydrochloride (9)

To a stirred solution of amine 1i (27 mg, mmol) in ethyl acetate (1 mL) was added ethereal HCl (1 M) in excess. A white solid immediately formed, the solvent was removed in vacuo, and the resulting solid was recrystallized from ethanol-ethyl acetate to yield an off white powder (9, 29 mg, quantitative): m.p. 169-170.5 EC; [α]²⁵_{D} 82.5E (c 0.73, CH₃OH); ¹H-NMR (300 MHz, CDCl₃) δ 3.07 (2H, m, —CH₂—), 3.70 (3H, s, OCH₃), 3.79 (3H, s, OCH₃), 4.27 (1H, m, alpha H), 5.93 (2H, s, —OCH₂O—), 6.37 (1H, d, J=1.2 Hz, ArH), 6.42 (1H, d, J=12.0 Hz, vinyl H), 6.46 (1H, d, J=12.6 Hz, vinyl H), 6.49 (1H, d, J=1.2 Hz, ArH), 6.75 (2H, d, J=8.4 Hz, 2×ArH), 6.90 (1H, d, J=8.1 Hz, ArH), 7.03 (1H, dd, J=8.1, 2.1 Hz, ArH), 7.09 (2H, d, J=8.7 Hz, 2×ArH), 7.92 (1H, d, J=1.5 Hz, ArH); Anal. calcd for C₂₆H₂₆ClN₂O₆: C, 59.32; H, 5.75; N, 5.33. Found: C, 59.53; H, 5.56; H, 5.26.

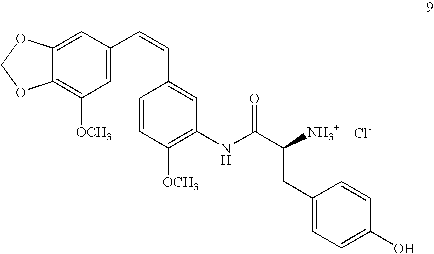

### 3,4-Methylenedioxy-5,4′-dimethoxy-3′-(O^{β}-tert-butyl-N^{α}-Boc-L-Asp)-amido-Z-stilbene (10)

To a stirred mixture of amine 1a (0.13 g, 0.43 mmol), O^{β}-tert-butyl-N^{α}-Boc-L-Asp (0.22 g, 0.76 mmol, 1.8 eq), and PyBroP (0.35 g, 0.76 mmol, 1.8 eq) in DCM (3 mL) at 0 EC under Ar was added DIPEA (0.21 mL, 1.2 mmol, 2.8 eq). The reaction mixture was stirred for 75 min at rt, and the solvent was removed in vacuo. The product was obtained by flash column chromatography (1:1, n-hexane:acetone) as an oil (10, 0.22 g, 88%): R_{f} 0.64 (1:1, n-hexane-acetone); [α]²⁴_{D} −13.0E (c 1.42, CHCl₃); ¹H-NMR (300 MHz, CDCl₃) δ 1.44 (9H, s, tBu), 1.49 (9H, s, tBu), 2.88 (2H, m, —CH₂—), 3.73 (3H, s, OCH₃), 3.84 (3H, s, OCH₃), 4.59 (1H, m, alpha H), 5.81 (1H, br), 5.92 (2H, s, —OCH₂O—), 6.38 (1H, d, J=12.6 Hz, vinyl H), 6.44 (1H, s, ArH), 6.46 (1H, d, J=12.6 Hz, vinyl H), 6.47 (1H, s, ArH), 6.70 (1H, d, J=8.1 Hz, ArH), 6.97 (1H, dd, J=8.4, 1.8 Hz, ArH), 8.28 (1H, d, J=2.4 Hz, ArH), 8.76 (1H, s); and HRMS calcd for C₃₀H₃₉N₂O₉ [M+H]⁺ 571.2655; found 571.2617. Anal. calcd for C₃₀H₃₈N₂O₉: C, 63.14; H, 6.71; N, 4.91. Found: C, 62.64; H, 7.00; N, 4.89.

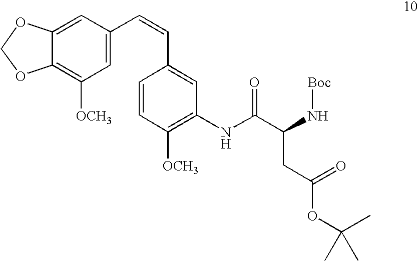

### Sodium 3,4-methylenedioxy-5,4′-dimethoxy-3′-(N^{α}-L-Asp)-amido-Z-stilbene (11)

To t-butyl ester 10 (0.13 g, 0.23 mmol) in DCM (1.5 mL) was added a mixture of TFA (0.71 mL, 9.6 mmol, 42 eq) and TES (0.30 mL, 1.8 mmol, 7.8 eq), and stirring was continued for 4 h under Ar. The solvents were removed in vacuo, and the TFA salt, obtained by Sephadex LH-20 column chromatography (solvent, MeOH), was placed in methanol (1 mL). Sodium methoxide (0.22 mg, 0.41 mmol, 1.8 eq) was added to the reaction mixture, and the white precipitate that formed was collected and reprecipitated from DCM-CH₃OH to give the product 11 as a colorless solid (62 mg, 62%): m.p. 168-170 EC; [α]²⁵_{D} +14.0E (c 1.18, CH₃OH); ¹H-NMR (300 MHz, CD₃OD) δ 2.62 (1H, m, —CH₂—), 2.77 (1H, m, —CH₂—), 2.78 (2H, br), 3.69 (3H, s, OCH₃), 3.87 (3H, s, OCH₃), 4.29 (1H, m, alpha H), 5.87 (2H, s, —OCH₂O—) 6.36 (1H, d, J=1.2 Hz, ArH), 6.40 (1H, d, J=12.0 Hz, vinyl H), 6.45 (1H, d, J=12.3 Hz, vinyl H), 6.50 (1H, d, J=0.9 Hz, ArH), 6.91 (1H, d, J=8.4 Hz, ArH), 7.02 (1H, dd, J=8.4, 1.8 Hz, ArH), 7.99 (1H, d, J=2.4 Hz, ArH), 8.52 (1H, s); Anal. calcd for C₂₁H₂₁N₂NaO₇H₂O: C, 55.51; H, 5.10; N, 6.16. Found: C, 55.81; H, 5.70; N, 6.16.

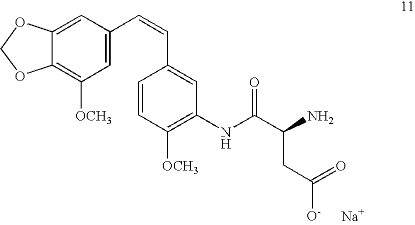

### 3,4-Methylenedioxy-5,4′-dimethoxy-3′-(N^{α}-Boc-L-Dap)-amido-Z-stilbene (13)

To a stirred mixture of amine 1a (51 mg, 0.17 mmol), N^{α}-Boc-L-Dap 12 (52 mg, 0.18 mmol, 1.1 eq),^{32,33} and PyBroP (87 mg, 0.19 mmol, 1.1 eq) at 0 EC under Ar was added DIPEA (65 μL, 0.37 mmol, 2.2 eq). The reaction mixture was stirred for 1.5 h at rt. DCM (10 mL) was added, and the mixture was washed with aqueous citric acid (10% by wt, 10 mL). The organic layer was dried with magnesium sulfate and concentrated in vacuo, and the residue was subjected to gravity column chromatography (8:1, DCM-EtOAc), resulting in the product 13 as a colorless oil (40 mg, 41%): R_{f} 0.24 (8:1, DCM:EtOAc); [α]²⁴_{D} −48.5E (c 1.2, CHCl₃); ¹³C-NMR (500 MHz, CDCl₃) δ 148.5, 146.9, 143.3, 134.2, 131.9, 130.1, 129.5, 128.8, 127.6, 123.8, 120.8, 109.6, 108.5, 103.0, 101.3, 58.7, 56.3, 55.8, 28.5; ¹H-NMR (300 MHz, CDCl₃) δ 1.30 (3H, d, J=6.9 Hz, CH₃), 1.46 (9H, s, Boc), 1.72 (1H, m, Pro), 1.90 (3H, m, Pro), 2.62 (1H, m), 3.23 (1H, m), 3.44 (1H, m), 3.49 (3H, s, OCH₃), 3.58 (1H, m), 3.74 (3H, s, OCH₃) 3.85 (3H, s, OCH₃), 3.90 (1H, m), 5.92 (2H, s, —OCH₂—), 6.38 (1H, d, J=12.3 Hz, vinyl H), 6.45 (1H, s, ArH), 6.46 (1H, d, J=12.0 Hz, vinyl H), 6.47 (1H, s, ArH), 6.69 (1H, d, J=8.7, ArH), 6.96 (1H, dd, J=8.4, 1.8 Hz, ArH), 8.30 (1H, d, J=2.4, ArH), 8.41 (1H, br); and HRMS calcd for C₃₁H₄₁N₂O₈ [M+H]⁺ 569.2863; found 569.2884. Anal. calcd for C₃₁H₄₀N₂O₈: C, 65.48; H, 7.09; N, 4.93. Found: C, 64.92; H, 7.42; N, 4.97.

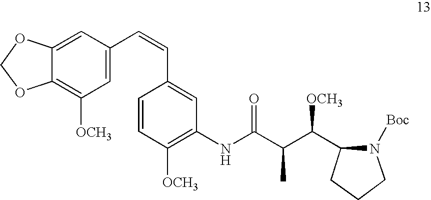

### 3,4-Methylenedioxy-5,4′-dimethoxy-3′-(N^{α}-L-Dap)-amido-Z-stilbene TFA salt (14)

To a stirred solution of amide 13 (0.15 g, 0.26 mmol) in DCM (0.65 mL) at 0 EC was added TFA (0.20 mL, 2.6 mmol, 10 eq). The reaction mixture was stirred for 20 min at rt. The product was obtained by gravity column chromatography (20:1, DCM:MeOH) as an oil (14, 69 mg, 46%): R_{f} 0.54 (8:1, DCM:MeOH); [α]²⁴_{D} −61.9E (c 0.88, CHCl₃; ¹³C-NMR (300 MHz, CDCl₃) δ 170.5, 148.1, 146.9, 142.8, 131.3, 129.6, 128.6, 126.3, 124.3, 120.5, 109.3, 108.1, 102.4, 100.8, 100.3, 80.2, 60.5, 58.6, 55.8, 55.3, 44.8, 41.5, 24.1, 23.4, 12.1; ¹H-NMR (300 MHz, CDCl₃) δ 1.27 (3H, d, J=6.9, CH₃), 1.98 (4H, m), 2.94 (1H, m), 3.33 (2H, m), 3.57 (3H, s, OCH₃), 3.73 (1H, s, OCH₃), 3.78 (1H, m), 3.85 (3H, s, OCH₃), 3.89 (1H, m), 5.91 (2H, s, —OCH₂O—), 6.38 (1H, d, J=12.6, vinyl H), 6.43 (3H, m, vinyl H, 2×ArH), 6.73 (1H, d, J=9.0, ArH), 7.00 (1H, dd, J=8.4, 1.8 Hz, ArH), 8.15 (1H, d, J=1.8 Hz, ArH), 8.35 (1H, s); and HRMS calcd for C₂₆H₃₃N₂O₆ [M+H]⁺ 469.2339; found 469.2374. Anal. calcd for C₃₀H₃₄F₆N₂O₁₀2H₂O: C, 49.18; H, 5.23; N, 3.82. Found: C, 49.48; H, 5.23; N, 4.09.

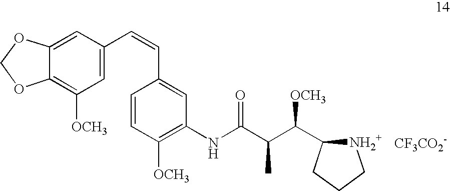

### 3,4-Methylenedioxy-5,4′-dimethoxy-3′-(Dov-Val-Dil-Dap)-amido-Z-stilbene (16)

To a stirred mixture of amide 14 (41 mg, 0.070 mmol), Dov-Val-Dil TFA salt 15 (62 mg, 0.11 mmol, 1.6 eq) and PyBroP (50 mg, 0.11 mmol, 1.6 eq) in DCM (0.5 mL) at 0 EC under Ar was added DIPEA (91 μL, 0.52 mmol, 7.4 eq). The reaction mixture was stirred 14 h at rt, and the solvent was removed in vacuo. The product was obtained by gravity column chromatography (1:1, n-hexane:acetone) as a colorless oil (16, 30 mg, 48%): R_{f} 0.34 (1:1, n-hexane-acetone); [α]²³_{D} −62E (c 1.05, CHCl₃); ¹H-NMR (300 MHz, CDCl₃) δ 0.79 (3H, t, J=6.9 Hz, CH₃), 0.91 (3H, d, J=6.6 Hz, CH₃), 0.94 (3H, d, J=6.6 Hz), 0.94 (3H, d, J=6.3 Hz, CH₃), 1.00 (3H, d, J=6.6 Hz, CH₃), 1.01 (3H, d, J=6.3 Hz, CH₃), 1.32 (3H, d, J=6.9 Hz, CH₃), 1.35 (2H, m), 1.80 (1H, m), 2.0 (3H, m), 2.31 (6H, s, 2×NCH₃), 2.34 (4H, m), 2.62 (1H, m), 3.00 (3H, s, NCH₃), 3.05 (1H, m) 3.08 (1H, s), 3.20 (1H, m), 3.29 (1H, m), 3.32 (3H, OCH₃), 3.34 (1H, m), 3.44 (1H, m), 3.45 (3H, s, OCH₃), 3.72 (3H, s. OCH₃), 3.84 (3H, s, OCH₃), 4.06 (1H, m), 4.12 (1H, m), 4.20 (1H, m), 4.79 (1H, m), 5.90 (2H, s, —OCH₂O—), 6.36 (1H, d, J=12.0 Hz, vinyl H), 6.44 (3H, m, vinyl H, 2×ArH), 6.68 (1H, d, J=8.7 Hz, ArH), 6.94 (1H, dd, J=8.1, 1.5 Hz, ArH), 8.27 (1H, d, J=1.8 Hz), 8.33 (1H, s); and HRMS calcd for C₄₈H₇₄N₅O₁₀ [M+H]⁺ 880.5435; found 880.5426. Anal. calcd for C₄₈H₇₃N₅O₁₀: C, 65.50; H, 8.36; N, 7.96. Found: C, 65.42; H, 8.74; N, 7.59.

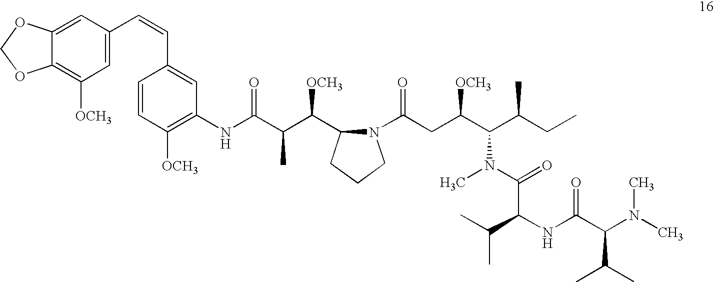

### Tubulin and Cancer Cell Procedures.

Bovine brain tubulin was prepared and the tubulin assembly and colchicine binding assays were performed as described previously. In the assembly assay, reaction mixtures contained 1.0 mg/mL (10 μM) tubulin and varying concentrations of potential inhibitors. In the colchicine binding assay reaction mixtures contained 0.1 mg/mL (1.0 μM) tubulin, 5.0 μM [³H]colchicine, and potential inhibitor as indicated. The vinblastine binding assay was performed as described previously for GTP binding by centrifugal gel filtration, except that a Beckman Allegra 6KR centrifuge equipped with a GH-3.8A swinging bucket rotor was used and the syringe-columns were centrifuged at 2,000 rpm (Ref. 38-41). Reaction mixtures contained 0.5 mg/mL (5.0 μM) tubulin, 5 μM [³H]vinblastine, potential inhibitor as indicated, 0.5 mM MgCl₂, 0.1 M 4-morpholineethanesulfonate (1.0 M stock solution adjusted to pH 6.9 with NaOH, and 4% (v/v) dimethyl sulfoxide. Incubation (30 min) and centrifugal gel filtration were performed at room temperature (20-22° C.).

Cytotoxicity assays were performed by the sulforhodamine B method, in which inhibition of formation of cellular protein is measured (Ref. 42). The mitotic index of MCF-7 breast cancer cells was determined as described previously (Ref. 27), except that cells were incubated in the presence of drug for 16 h prior to addition of the DNA stain.

The preceding technological disclosure describes illustrative embodiments of these new stilbene derivative compounds and their proposed use and does not intend to limit the present invention to these precise embodiments. Further, any changes and/or modifications, which may be obvious by one with ordinary skill in the related art, including but not limited to pharmaceutical salt derivatives or non-functional changes of the disclosed chemical compounds, are intended to be included within the scope of the invention.

### REFERENCES AND NOTES

1. Pettit, G. R.; Moser, B. R.; Boyd, M. R.; Schmidt, J. M.; Pettit, R. K.; Chapuis, J. C. Antineoplastic Agents 460. Synthesis of Combretastatin A-2 Prodrugs. Anti-Cancer Drug Des., 2002, 16, 185-194.
2. Kanthou, C.; Tozer, G. M. The Tumor Vascular Targeting Agent Combretastatin A-4-Phosphate Induces Reorganization of the Actin Cytoskeleton and Early Membrane Blebbing in Human Endothelial Cells. Blood 2002, 99, 2060-2069.
3. Beauregard, D. A.; Pedley, R. B.; Hill, S. A. Brindle, K. M. Differential Sensitivity of Two Adenocarcinoma Xenografts to the Anti-Vascular Drugs Combretastatin A4 Phosphate and 5,6-Dimethylxanthenone-4-Acetic Acid, Assessed Using MRI and MRS. NMR in Biomed. 2002, 15, 99-105.
4. Eikesdal, H. P.; Landuyt, W.; Dahl, O. The Influence of Combretastatin A-4 and Vinblastine on Interstitial Fluid Pressure in BT4An Rat Gliomas. Cancer Lett. 2002, 178, 209-217.
5. Li, L.; Rojiani, A. M.; Siemann, D. W. Preclinical Evaluations of Therapies Combining the Vascular Targeting Agent Combretastatin A-4 Disodium Phosphate and Conventional Anticancer Therapies in the Treatment of Kaposi's Sarcoma. Acta Oncol. 2002, 41, 91-97.
6. Boehle, A. S.; Sipos, B.; Kliche, U.; Kalthoff, H.; Dohrnann, P. Combretastatin A-4 Prodrug Inhibits Growth of Human Non-Small Cell Lung Cancer in a Murine Xenotransplant Model. Ann. Thoracic Surg. 2001, 71, 1657-1665.
7. Dowlati, A.; Robertson, K.; Cooney, M.; Petros, W. P.; Stratford, M.; Jesberger, J.; Rafie, N.; Overmoyer, B.; Makkar, V.; Stambler, B.; Taylor, A.; Waas, J.; Lewin, J. S.; McCrae, K. R.; Remick, S. C. A Phase I Pharmacokinetic and Translational Study of the Novel Vascular Targeting Agent Combretastatin A-4 Phosphate on a Single-Dose Intravenous Schedule in Patients with Advanced Cancer. Cancer Res. 2002, 62, 3408-3416.
8. Remick, S. C.; Dowlati, A.; Robertson, K.; Spiro, T.; Connell, C.; Levitan, N.; Stratford, M. Phase I Pharmacokinetics Study of Single Dose Intravenous (IV) Combretastatin A-4 Prodrug (CA4P) in Patients (Pts) with Advanced Cancer. In Molecular Targets and Cancer Therapeutics Discovery, Development, and Clinical Validation. Proceedings of the AACR-NCI-EORTC International Congress, Washington, D.C.: 1999:4.
9. Rustin, G. J. S.; Galtraith, S. M.; Taylor, N. J.; Maxwell, R.; Tozer, G.; Baddeley, H.; Wilson, L.; Prise V. Combretastatin A-4 Phosphate (CA4P) Selectively Targets Vasculature in Animal and Human Tumors. In Molecular Targets and Cancer Therapeutics Discovery, Development, and Clinical Validation. Proceedings of the AACR-NCI-EORTC International Congress, Washington, D.C.: 1999:4.
10. Pettit, G. R.; Temple, C.; Narayanan, V. L.; Varma, R.; Simpson, M. M.; Boyd, M. R.; Rener, G. A.; Bansal, N. Antineoplastic Agents 322. Synthesis of Combretastatin A-4 Prodrugs. Anti-Cancer Drug Des. 1995, 10, 299-309.
11. Pettit, G. R.; Minardi, M. D.; Boyd, M. R.; Pettit, R. K. Antineoplastic Agents 463. Synthesis of Combretastatin A-3 Diphosphate Prodrugs. Anti-Cancer Drug Des. 2000, 15, 397-404.
12. Pettit, G. R.; Lippert, J. W. Antineoplastic Agents 429. Synthesis of the Combretastatin A-1 and Combretastatin B-1 Prodrugs. Anti-Cancer Drug Des. 2000, 15, 203-216.
13. 13. Pettit, G. R.; Rhodes, M. R. Antineoplastic Agents 389. New Syntheses of the Combretastatin A-4 Prodrug. Anti-Cancer Drug Des. 1998, 13, 183-191.
14. Chakravarty, P. K.; Greenlee, W. J.; Parsons, W. H.; Patchett, A. A.; Combs, P.; Roth, A.; Busch, R. D.; Mellin, T. N. (3-Amino-2oxoalkyl) phosphonic Acids and their Analogues as Novel Inhibitors of D-Alanine:D-Alanine Ligase. J. Med. Chem. 1989, 32, 1886-1890.
15. Peffit G. R.; Kamano Y.; Herald C. L.; Tuinman A. A.; Boettner F. E.; Kizu H.; Schmidt J. M.; Baczynsky, L.; Tomer, K. B.; Bontems, R. J. Antineoplastic Agents 136. The Isolation and Structure of a Remarkable Marine Animal Antineoplastic Constituent-Dolastatin 10. J. Amer. Chem. Soc. 1987, 109, 6883-6885.
16. Pettit, G. R. The Dolastatins. In Herz, W.; Kirby, G. W.; Moore, R. E.; Steglich, W.; Tamm, Ch., eds. Progress in the Chemistry of Organic Natural Products 70. New York: Springer Wien; 1997:1-79.
17. Pettit, G. R. Evolutionary Biosyntheseis of Anticancer Drugs. In: Ojima, I.; Vite, G. D.; Altmann, K-H., eds. Anticancer Agents, Frontiers in Cancer Chemotherapy. Washington, D.C.: American Chemical Society; 2001:16-42.
18. Dang, L. H.; Bettegowda, C.; Huso, D. L.; Kinzler, K. W.; Vogelstein, B. Combination Bacteriolytic Therapy for the Treatment of Experimental Tumors. Prod. Natl. Acad. Sci. 2001, 98, 15155-15160.
19. Thamm, D. H.; MacEwen, E. G.; Phillips, B. S.; Hershey, A. E.; Burgess, K. M.; Pettit, G. R.; Vail D. M. Preclinical Study, of Dolastatin-10 in Dogs with Spontaneous Neoplasia. Cancer Chemother. Pharmacol. 2002, 49, 251-255.
20. Margolin, K.; Longmate, J.; Synold, T. W.; Gandara, D. R.; Weber, J.; Gonzalez, R.; Johansen, M. J.; Newman, R.; Baratta, T.; Doroshow, J. H. Dolastatin-10 in Metastatic Melanoma: A Phase II Phannokinetic Trial of the California Cancer Consortium. Invest. New Drugs 2001, 19, 335-340.
21. Vaishampayan, U.; Glode, M.; Du, W.; Kraft, A.; Hudes, G.; Wright, J.; Hussain, M. Phase II Study of Dolastatin-10 in Patients with Hormone-Refractory Metastatic Prostate Adenocarcinoma. Clin. Cancer Res. 2000, 6, 4205-4208.
22. Krug, L. M.; Miller, V. A.; Kalemkerian, G. P.; Kraut, M. J.; Ng, K. K.; Heelan, R. T.; Pizzo, B. S.; Perez, W.; McClean, N.; Kris, M. G. Phase II study of Dolastatin-10 in Patients with Advanced Non-Small-Cell Lung Cancer. Ann. Oncol. 2000, 11, 227-228.
23. Pettit, R. K.; Pettit, G. R.; Hazen, K. C. Specific Activities of Dolastatin 10 and Peptide Derivatives Against Cryptococcus neoformans. Antimicrob. Agents Chemother. 1998, 42, 2961-2965.
24. Woyke, T.; Pettit, G. R.; Winkelmann, G.; Pettit, R. K. In vitro Activities and Postantifungal Effects of the Potent Dolastatin 10 Derivative Auristatin PHE. Antimicrob. Agents Chemother. 2001, 45, 3580-3584.
25. Pettit, G. R.; Srirangam J. K.; Barkoczy J.; Williams M. D.; Boyd M. R.; Hamel E.; Pettit R. K.; Hogan F.; Bai R.; Chapuis J-C.; McAllister S. C.; Schmidt J. M. Antineoplastic Agents 365. Dolastatin 10 SAR Probes. Anti-Cancer Drug Des. 1998, 13, 243-277.
26. Pettit, G. R.; Srirangam, J. K.; Barkoczy, J.; Williams, M. D.; Durkin, K. P. M.; Boyd, M. R.; Bai, R. L.; Hamel, E.; Schmidt, J. M.; Chapuis, J. C. (1995) Antineoplastic Agents 337. Synthesis of Dolastatin 10 Structural Modifications. Anti-Cancer Drug Des. 1995, 10, 529-544.
27. Pettit, G. R.; Anderson, C. R.; Herald, D. L.; Jung, M. K.; Lee, D. J.; Hamel, E.; Pettit, R. K. Antineoplastic Agents 487. Synthesis and Biological Evaluation of the Antineoplastic Agent 3,4-Methylenedioxy-5,4¹-Dimethoxy-3¹-Amino-Z-Stilbene and Derived Amino Acid Amides. J. Med. Chem. 2003, 46, 525-531.
28. Coste, J.; Dufour, M-N.; Pantaloni, A.; Bertrand, C. Brop: A New Reagent for Coupling N-Methylated Amino Acids. Tetrahedron Lett. 1990, 31, 669-672.
29. Lott, R. S.; Chauhan, V. S.; Stammer, C. H. Trimethylsilyl Iodide As a Peptide Deblocking Agent. J. Chem. Soc., Chem. Commun. 1979, 495-496; 1979.
30. Ho, T-L.; Olah, G. A. Cleavage of Esters and Ethers with Iodotrimethylsilane. Angew. Chem. Intl. Ed. (Engl.) 1976, 15, 774-775.
31. Mehta, A.; Jaouhari, R.; Benson, T. J.; Douglas, K. T. Improved Efficiency and Selectivity in Peptide Synthesis: Use of Triethylsilane as a Carbocation Scavenger in Deprotection of t-Butyl Esters and t-Butoxycarbonyl-Protected Sites. Tetrahedron Lett. 1992, 33, 5441-5444; 1992.
32. Pettit, G. R.; Srirangam, J. K.; Singh, S. B.; Williams, M. D.; Herald, D. L.; Barkoczy, J.; Kantoci, D.; Hogan, F. Dolastatins 24. Synthesis of (−)-Dolastatin 10. X-Ray Molecular Structure of NN-dimethylvalyl-valyl-dolaisoleuine tert-butyl ester. J. Chem. Soc., Perkin Trans. 1 1996, 859-863.
33. Pettit, G. R.; Burkett, D. D.; Barkoczy, J.; Breneman, G. L.; Pettit, W. E. The Dolastatins 18. Sterospecific Synthesis of Dolaproine. Synthesis 1996, 719-725.
34. Bai, R.; Pettit, G. R.; Hamel, E. Dolastatin 10, a Powerful Cytostatic Peptide Derived from a Marine Animal: Inhibition of Tubulin Polymerization Mediated Through the Vinca Alkaloid Binding Domain. Biochem. Pharmacol. 1990, 39, 1941-1949.
35. Bai, R.; Pettit, G. R.; Hamel, E. Binding of Dolastatin 10 to Tubulin at a Distinct Site for Peptide Antimitotic Agents Near the Exchangeable Nucleotide and Vinca Alkaloid Sites. J. Biol. Chem. 1990, 265, 17141-17149.
36. Lin, C. M.; Singh, S. B.; Chu, P. S.; Dempcy, R. O.; Schmidt, J. M.; Pettit, G. R.; Hamel, E. Interactions of Tubulin with Potent Natural and Synthetic Analogs of the Antimitotic Agent Combretastatin, a Structure-Activity Study. Mol. Pharmacol. 1988, 34, 200-208.
37. Lin, C. M.; Ho, H. H.; Pettit, G. R.; Hamel, E. The Antimitotic Natural Products Combretastatin A-4 and Combretastatin A-2: Studies on the Mechanism of their Inhibition of the Binding of Colchicine to Tubulin. Biochemistry 1989, 28, 6984-6991.
38. Hamel, E.; Lin, C. M. Separation of Active Tubulin and Microtubule-Associated Proteins by Ultracentrifugation and Isolation of a Component Causing the Formation of Microtubule Bundles. Biochemistry 1984, 23, 4173-4184.
39. Hamel, E. Evaluation of Antimitotic Agents by Quantitative Comparisons of their Effects on the Polymerization of Purified Tubulin. Cell Biochem. Biophys. 2003, 38, 1-21.
40. Verdier-Pinard, P.; Lai, J.-Y.; Yoo, H.-D.; Yu, J.; Marquez, B.; Nagle, D. G.; Nambu, M.; White, J. D.; Falck, J. R.; Gerwick, W. H.; Day, B. W.; Hamel, E. Structure-Activity Analysis of the Interaction of Curacin A, the Potent Colchicine Site Antimitotic Agent, with Tubulin and Effects of Analogs on the Growth of MCF-7 Breast Cancer Cells. Mol. Pharmacol. 1998, 53, 62-76.
41. Hamel, E.; Lin, C. M.; Guanosine 5′-O-(3-thiotriphosphate), a Potent Nucleotide Inhibitor of Microtubule Assembly. J. Biol. Chem. 1984, 259, 11060-11069.
42. Skehan, P.; Storeng, R.; Scudiero, D.; Monks, A.; McMahon, J.; Vistica, D.; Warren, J. T.; Bokesch, H.; Kenney, S.; Boyd, M. R. New Colorimetric Cytotoxicity Assay for Anticancer-Drug Screening. J. Nad. Cancer Inst. 1990, 82, 1107-1112.
43. NCCLS. Methods for Dilution Antimicrobial Susceptibility Tests for Bacteria that Grow Aerobically; Approved Standard-Fifth Edition. NCCLS Document M7-A5; 2000.
44. NCCLS. Reference Method for Broth Dilution Antifungal Susceptibility Testing of Yeasts; Approved Standard-Second Edition. NCCLS Document M27-A2; 2002.

What is claimed is:

1. A compound of the formula 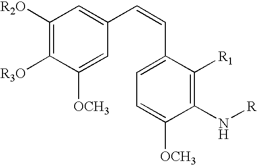
or a salt thereof, wherein
R is Dap, Dap-Dil, Dap-Dil-Val, or Dap-Dil-Val-Dov;
R₁ is H, OH, or PO₃Na₂; and
R₂ and R₃ are jointly —CH₂— or each independently H, OH, CH₃, or PO₃Na₂.

2. The compound of claim 1, wherein R is Dap.

3. The compound of claim 2, wherein R₁ is H.

4. The compound of claim 3, wherein the compound is formula

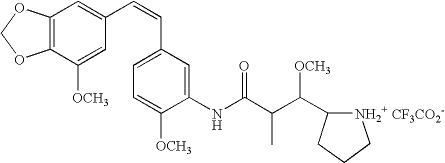

5. The compound of claim 1, wherein R is Dap-Dil-Val-Dov.

6. The compound of claim 5, wherein R₁ is H.

7. The compound of claim 6, wherein the compound is of the formula

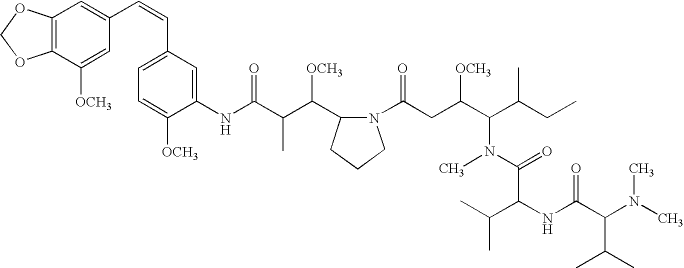

8. A composition comprising at least one compound of claim 1 and a pharmaceutically acceptable carrier.

9. The composition of claim 8, wherein the compound is in a therapeutically effective amount sufficient to inhibit cancer cell growth.

10. The composition of claim 8, wherein the compound has anticryptococcal or antibacterial activity and is in a therapeutically effective amount sufficient to inhibit the growth of a parasitic microbe.

11. The composition of claim 10, wherein the compound inhibits the growth of at least one of the following: Neisseria gonorrhoeae; Enterococcus faecalis; Streptococcus pneumoniae; and Cryptococcus neoformans.

12. A compound of the formula 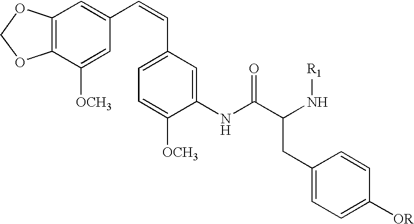
and salts thereof, wherein
R and R₁ are independently H or P(O)(OH).

13. The compound of claim 12, wherein the compound is of the formula

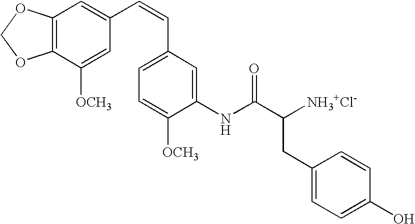

14. A method of inhibiting cancer cell growth in a host inflicted therewith comprising administering to the host the compound of claim 13 in a therapeutically effective amount sufficient to inhibit growth of the cancer cells in the host.

15. A compound of the formula

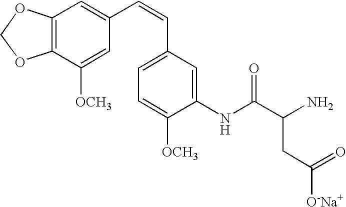

16. A method of inhibiting cancer cell growth in a host inflicted therewith comprising administering to the host the compound of claim 15 is in a therapeutically effective amount sufficient to inhibit cancer cell growth in the host.

17. A method of inhibiting cancer cell growth in a host inflicted therewith comprising administering to the host a compound of formula 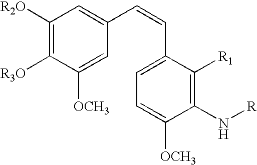
or a salt thereof and is in a therapeutically effective amount sufficient to inhibit growth of the cancer cells in the host, wherein
R is Dap, Dap-Dil, Dap-Dil-Val, or Dap-Dil-Val-Dov;
R₁ is H, OH, or PO₃Na₂; and
R₂ and R₃ are jointly —CH₂— or each independently H, OH, CH₃, or PO₃Na₂.

18. The method of claim 17, wherein the R is Dap-Dil-Val-Dov.

19. The method of claim 18, wherein R₁ is H.

20. The method of claim 17, wherein the compound is in a pharmaceutically acceptable carrier and the host is a human.

21. The method of claim 17, wherein the cancer inhibited is selected from the group consisting of: leukemia, pancreas, breast, CNS, lung-NSC, colon, or prostate cancer.

22. A method of inhibiting microbial growth in a host inflicted therewith comprising administering to the host a compound of formula 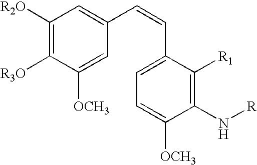
or a salt thereof and is in a therapeutically effective amount sufficient to inhibit the microbial growth, wherein
R is Dap, Dap-Dil, or Dap-Dil-Val;
R₁ is H, OH, or PO₃Na₂; and
R₂ and R₃ are jointly —CH₂— or each independently H, OH, CH₃, or PO₃Na₂.

23. The method of claim 22, wherein R is Dap.

24. The method of claim 23, wherein R₁ is H.

25. The method of claim 22, wherein the compound inhibits the growth of one of the following: Neisseria gonorrhoeae; Enterococcus faecalis; Streptococcus pneumoniae; and Cryptococcus neoformans.

26. The method of claim 22, wherein the compound is in a pharmaceutically acceptable carrier and the host is a human.
